(19) United States
(12) Patent Application Publication
(10) Pub. No.: US 2026/0098830 A1
(43) Pub. Date: Apr. 9, 2026

(54) ELECTRODE AND METHOD FOR MAKING AN ELECTRODE

(71) Applicant: SAINT-GOBAIN PERFORMANCE PLASTICS CORPORATION, Malvern, PA (US)

(72) Inventors: Shiwei LIU, Stevenson Ranch, CA (US); Sabrina D. OROZCO, San Diego, CA (US); Yi JIANG, Shrewsbury, MA (US); John V. VO, San Diego, CA (US); Robrecht MOERKERKE, Kortrijk (BE); Sethumadhavan RAVICHANDRAN, San Diego, CA (US); Jean-Thomas FONNE, Benfeld (FR); Quinten VAN DEN BERGHE, Brakel (BE)

(21) Appl. No.: 19/350,865

(22) Filed: Oct. 6, 2025

Related U.S. Application Data
(60) Provisional application No. 63/704,102, filed on Oct. 7, 2024.

(51) Int. Cl. G01N 27/327; G01N 33/66; H01B 1/02; H01B 1/08; H01B 3/30

(52) U.S. Cl. CPC G01N 27/3272; G01N 33/66; H01B 1/02; H01B 1/08; H01B 3/30

(57) ABSTRACT

An electrochemical biosensor electrode includes a substrate; a base layer adjacent to the substrate, the base layer including an inert, non-noble metal; and a top layer adjacent to the base layer, the top layer including a noble metal, wherein the top layer has a thickness of not greater than 15 nanometers (nm).

### CROSS-REFERENCE TO RELATED APPLICATION(S)

This application claims priority under 35 U.S.C. § 119 (c) to U.S. Provisional Patent Application No. 63/704,102 entitled “ELECTRODE AND METHOD FOR MAKING AN ELECTRODE”, filed Oct. 7, 2024, by Shiwei LIU et al., which is assigned to the current assignee hereof and is incorporated herein by reference in its entirety.

### FIELD OF THE DISCLOSURE

The present disclosure relates to an electrode, and more particularly to, a thin film electrode for biosensor applications.

### RELATED ART

Electrochemical glucose biosensors generally include two electrodes with at least one of the electrodes having a metallic layer. This metallic layer can be chosen for a variety of factors, including a combination of the metals resistivity and electrochemical inertness. However, metals that generally meet such requirements for use in electrochemical glucose biosensors can be expensive and any reduction in their quantity or quality can degrade the overall performance of the biosensors. So, there is a continuing need for an electrode design that can be manufactured in a cost effective manner while still maintaining or improving overall performance.

### SUMMARY

According to a first aspect, an electrochemical biosensor electrode includes a substrate; a base layer adjacent to the substrate, the base layer including an inert, non-noble metal, the inert, non-noble metal comprising nickel (Ni), chromium (Cr), iron (Fc), manganese (Mn), molybdenum (Mo), an alloy thereof, or combination thereof; and a top layer adjacent to the base layer, the top layer including a noble metal, wherein the top layer has a thickness of not greater than 15 nanometers (nm).

According to another aspect, an electrode may include a substrate; a base layer adjacent to the substrate, the base layer including an inert, non-noble metal, the inert, non-noble metal comprising nickel (Ni), chromium (Cr), iron (Fe), manganese (Mn), molybdenum (Mo), an alloy thereof, or combination thereof; an intermediate layer adjacent to the base layer, the intermediate layer including an inert, non-noble metal oxide; and a top layer adjacent to the intermediate layer, wherein the top layer includes a noble metal having a thickness of not greater than 15 nanometers (nm).

According to yet another aspect, a method of forming an electrochemical biosensor electrode including: providing a substrate; depositing a base layer on the substrate, wherein the base layer includes an inert, non-noble metal, the inert, non-noble metal comprising nickel (Ni), chromium (Cr), iron (Fe), manganese (Mn), molybdenum (Mo), an alloy thereof, or combination thereof; optionally forming an intermediate layer on the base layer, wherein the intermediate layer including an inert non-noble metal oxide; and depositing a top layer adjacent to the base layer, the top layer including a noble metal, the top layer having a thickness of not greater than 15 nanometers (nm).

### BRIEF DESCRIPTION OF THE DRAWINGS

Embodiments are illustrated by way of example and are not limited by the accompanying figure.

FIG. 1 includes an illustration of an electrode according to embodiments described herein.

FIG. 2 includes a graphical depiction of Cyclic Voltammetry (CV) scans of an exemplary conductive composite film.

FIG. 3 includes a graphical depiction of Cyclic Voltammetry (CV) scans of an exemplary conductive composite film.

FIG. 4 includes a graphical depiction of Cyclic Voltammetry (CV) scans of an exemplary conductive composite film.

FIG. 5 includes a graphical depiction of Cyclic Voltammetry (CV) scans of exemplary conductive composite films.

FIG. 6 includes a graphical depiction of Differential Pulse Voltammetry (DPV) scans of exemplary conductive composite films.

FIG. 7 includes a graphical depiction of Differential Pulse Voltammetry (DPV) scans of exemplary conductive composite films.

FIG. 8 includes a graphical depiction of scratching resistance of exemplary conductive composite films.

FIG. 9 includes a graphical depiction of SIMS analysis results of oxidation of an interface between layers of exemplary conductive composite films.

FIG. 10 includes a graphical depiction of Differential Pulse Voltammetry (DPV) scans of exemplary conductive composite films.

FIG. 11 includes a graphical depiction of scratching resistance of exemplary conductive composite films.

Skilled artisans appreciate that elements in the figures are illustrated for simplicity and clarity and have not necessarily been drawn to scale. For example, the dimensions of some of the elements in the figures can be exaggerated relative to other elements to help to improve understanding of embodiments of the invention.

### DETAILED DESCRIPTION

The following description in combination with the figures is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other embodiments can be used based on the teachings as disclosed in this application.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but can include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item can be used in place of a single item. Similarly, where more than one item is described herein, a single item can be substituted for that more than one item.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and can be found in textbooks and other sources within the electrode and biosensor arts.

Embodiments described herein are generally directed to an electrochemical biosensor electrode or a method of forming an electrochemical biosensor electrode where the electrode includes a substrate, a base layer adjacent to the substrate, and a top layer adjacent to the base layer. The base layer may include an inert, non-noble metal. The top layer may include a noble metal, the top layer having a thickness of not greater than 15 nanometers.

In certain embodiments, an electrochemical biosensor electrode formed according to embodiments described herein can be a thin film electrode. In a particular embodiment, the thin film electrode can be for use in biosensors or biosensor test strips that measure the glucose level in a sample, such as, a blood sample.

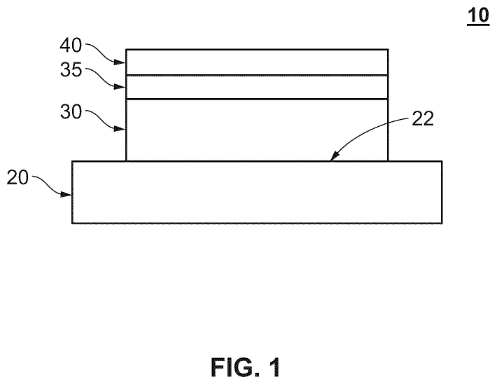

FIG. 1 includes an illustration showing the configuration of an electrode 10 formed according to embodiments described herein. As shown in FIG. 1, the electrode 10 may include a substrate 20, a base layer 30 and a top layer 40. The electrode 10 may include an optional intermediate layer 35, disposed between the base layer 30 and the top layer 40. The base layer 30 can be disposed adjacent to the substrate 20, such as directly contacting the substrate 20. Further, the top layer 40 can be disposed adjacent to the base layer 30. When present, the optional intermediate layer 35 is disposed directly on the base layer 30 with the top layer 40 disposed directly on the optional intermediate layer 35. When the optional intermediate layer 35 is not present, the top layer 40 directly contacts the base layer 30.

According to certain embodiments, the substrate 20 may be constructed out of any material suitable for the substrate of an electrode. According to certain embodiments, the material forming the substrate 20 can include a polymer, a flexible polymer, or a transparent polymer. Suitable polymers can include, for example, polycarbonate, polyacrylate, polyester, polyethylene terephthalate (PET), polyethylene naphthalate (PEN), cellulose triacetated (TCA or TAC), polyurethane (PU), or any combination thereof. In a particular embodiment, the substrate 20 may be polyethylene terephthalate (PET). In an embodiment, the substrate can be a glass substrate, such as a transparent glass substrate.

According to an embodiment, the substrate 20 may have a particular thickness suitable for an electrode. For example, the substrate 20 may have a thickness of at least about 25 microns, at least about 30 microns, at least about 35 microns, at least about 40 microns, at least about 45 microns, or at least about 50 microns. In an embodiment, the substrate 20 may have a thickness of not greater than about 350 microns, such as, not greater than about 325 microns, not greater than about 300 microns, or not greater than about 275 microns. It will be appreciated that the substrate 20 may have a thickness within a range between any of the minimum and maximum values noted above. It will be further appreciated that the substrate 20 may have a thickness of any value between any of the minimum and maximum values noted above. For example, the substrate can have a thickness in a range of from about 25 microns to about 350 microns or from about 40 microns to about 300 microns. In a particular embodiment, the substrate can have a thickness in a range of from about 100 microns to about 300 microns.

According to still other embodiments and as shown in FIG. 1, a surface 22 of the substrate 20 that is adjacent to base layer 30 can be treated to improve adhesion between the substrate 20 and base layer 30. Any treatment of the surface 22 to improve the adhesion is envisioned. For example, treating the surface 22 of the substrate 20 can include a laser treatment, a plasma treatment, a chemical treatment, or combination thereof. In another embodiment, the surface 22 is not treated.

According to a particular embodiment, the base layer 30 may contain one or more of the following materials and the one or more materials contained in the base layer 30 can have one or more, or even all, of the following characteristics.

According to certain embodiments, the base layer 30 may be referred to as a film. According to yet another embodiment, the base layer 30 may be referred to as a thin film.

In an embodiment, the base layer 30 may include an inert, non-noble metal. Any inert, non-noble metal is envisioned and includes, for example, nickel (Ni), chromium (Cr), iron (Fc), manganese (Mn), molybdenum (Mo), an alloy thereof, or combination thereof. In a more particular embodiment, the inert, non-noble metal is an alloy of a combination of the inert, non-noble metals, such as an alloy of chromium and nickel, a stainless steel alloy (i.e., a combination of the alloys of at least iron, chromium, and nickel), or combination thereof. According to still other embodiments, the base layer 30 may consist essentially of an alloy of chromium and nickel, a combination of the alloys of iron, chromium, and nickel, or a combination thereof. As used herein the phrase “consists essentially” refers to including at least 85 atomic % of a given material. According to still other embodiments, the base layer 30 may consist of the stainless steel alloy, the alloy of chromium and nickel, or combination thereof.

In an embodiment, the base layer 30 may have a particular thickness. For example, the base layer 30 may have a thickness of at least about 5 nm, such as, at least about 10 nm, at least about 15 nm, at least about 20 nm, at least 25 nm, at least 30 nm, at least 35 nm, or at least about 40 nm. In an embodiment, the base layer 30 may have a thickness of not greater than about 200 nm, such as, not greater than about 190 nm, not greater than about 180 nm, not greater than about 170 nm, not greater than 160 nm, or not greater than about 150 nm. It will be appreciated that the thickness of the base layer 30 may be any value within a range between any of the minimum and maximum values noted above. It will be further appreciated that the thickness of the base layer 30 may be any value between any of the minimum and maximum values noted above.

According to certain embodiments, the top layer 40 may be referred to as a film. According to yet other embodiments, the top layer 40 may be referred to as a thin film.

In an embodiment, the top layer 40 may include any reasonable noble metal such as, for example, gold, palladium, platinum, iridium, ruthenium, an alloy, or combination thereof. In an embodiment, the top layer 40 may consist essentially of gold, palladium, or combination thereof. According to still other embodiments, the top layer 40 may consist of gold, palladium or combination thereof. In a particular embodiment, the top layer may further include at least one trace element. For instance, the trace element may include argon, krypton, xenon, neon, nitrogen, or combination thereof. In an embodiment, the trace element is krypton. In a particular embodiment, the trace element is present at an amount of at least 1×10⁵ atoms/cm³, such as at least 1×10¹⁰ atoms/cm³. In an embodiment, the top layer 40 may consist essentially of the noble metal and a trace element, such as gold and krypton. According to still other embodiments, the top layer 40 may consist of gold and krypton.

According to still other embodiments, the top layer 40 may have a particular thickness. For example, the top layer 40 may have a thickness of at least about 2 nm, such as, at least about 3 nm, at least about 4 nm, or at least about 5 nm. According to still other embodiments, the top layer 40 may have a thickness of not greater than about 15 nm, such as, not greater than about 14 nm, not greater than about 13 nm, not greater than 12 nm, or not greater than about 11 nm. It will be appreciated that the thickness of the top layer 40 may be any value within a range between any of the minimum and maximum values noted above. It will be further appreciated that the thickness of the top layer 40 may be any value between any of the minimum and maximum values noted above. Although not to be bound by theory, the thickness of the noble metal of the top layer 40 is sufficiently thin to reduce the cost and amount of noble metal used, but desirably thick enough to deter the diffusion of underlayers and maintain desirable electrochemical performance of the top layer 40.

According to still other embodiments, the electrode 10 includes an optional intermediate layer 35. The intermediate layer 35 may include an inert, non-noble metal oxide. In an embodiment, the intermediate layer 35 is formed via the oxidation of the inert, non-noble metal of the base layer 30. In an embodiment, the intermediate layer 35 may include an oxide of: nickel (Ni), nickel alloy, chromium (Cr), chromium alloy, iron (Fe), iron alloy, manganese (Mn), manganese alloy, molybdenum (Mo), molybdenum alloy, or combination thereof. In an embodiment, the inert, non-noble metal oxide of the intermediate layer 35 includes an oxide of stainless steel. According to still other embodiments, the intermediate layer 35 may consist essentially of a nickel oxide, a chromium oxide, an iron oxide, or combination thereof. In a more particular embodiment, the intermediate layer 35 may consist of the nickel oxide, chromium oxide, and iron oxide.

According to still other embodiments, the intermediate layer 35 may have a particular thickness. For example, the intermediate layer 35 may have a thickness of at least about 0.5 nm, such as, at least about 0.75 nm, at least about 1.0 nm, at least about 1.25 nm, at least about 1.5 nm, at least 1.75, or at least about 2.0 nm. According to still other embodiments, the intermediate layer 35 may have a thickness of not greater than about 5.0 nm, such as, not greater than about 4.75, not greater than about 4.5 nm, not greater than 4.25, or not greater than about 4.0 nm. It will be appreciated that the thickness of the intermediate layer 35 may be any value within a range between any of the minimum and maximum values noted above. It will be further appreciated that the thickness of the intermediate layer 35 may be any value between any of the minimum and maximum values noted above.

In an embodiment, the intermediate layer 35 may have barrier properties. Although not being bound by theory, the intermediate layer 35 may be present to deter or prevent the oxidation of the inert, non-noble metal of the base layer 30. In an embodiment, the intermediate layer 35 may assist in preventing the diffusion of inert, non-noble metal atoms of the base layer 30 into the noble metal of the top layer 40. In particular, the intermediate layer 35 may increase the overall lifetime of the electrode 10. For instance, diffused inert, non-noble metal atoms are typically susceptible to oxidation in atmosphere, which may negatively impact electrochemical kinetics of the electrode 10. In an embodiment, the intermediate layer 35 has a desirable thickness to deter the diffusion of inert, non-noble metal atoms but is sufficiently thin enough to provide desirable conductivity through the electrode.

According to still other embodiments, the electrode 10 can include additional layers. For example, the electrode 10 can include an intermediate layer or intermediate layers (not shown in FIG. 1) disposed between one or more of the substrate 20, the base layer 30, the optional intermediate layer 35, and the top layer 40.

According to yet other embodiments, the electrode 10 formed as described herein may be an inert electrode, such as, an inert thin film electrode.

According to still other embodiments, the electrode 10 may have a particular sheet resistance. For example, the electrode 10 may have a sheet resistance of at least about 2.0 Ohm/sq, such as at least about 2.2 Ohm/sq, or at least about 2.4 Ohm/sq. According to still other embodiments, the electrode 10 may have a sheet resistance of not greater than about 30 Ohm/sq, such as, not greater than about 25 Ohm/sq, not greater than about 20 Ohm/sq, not greater than about 15 Ohm/sq, or not greater than about 10 Ohm/sq. It will be appreciated that the sheet resistance of the electrode 10 may be any value within a range between any of the minimum and maximum values noted above. It will be further appreciated that the sheet resistance of the electrode 10 may be any value between any of the minimum and maximum values noted above.

In certain embodiments, the electrode 10 can be an electrochemical biosensor electrode, such as, for example, an electrochemical biosensor electrode that can measure the glucose level of a sample, such as, a blood sample. In a particular embodiment, the electrode 10 can contain a layer comprising a chemical reagent (not shown in FIG. 1), such as a reagent containing an enzyme, a mediator, an indicator, or any combination thereof. In a particular embodiment, a chemical reaction with glucose takes place on the surface of the electrode 10. For example, glucose can be indirectly degraded by the electrode 10 by first reacting with an enzyme to form a subproduct and the electrode 10 is reactive with the subproduct.

According to still other embodiments, the electrode 10 can be part of a biosensor, such as, a biosensor test strip adapted to measure the level of glucose in a sample, such as, a blood sample. In certain embodiments, the test strip can include a working electrode and a counter electrode and the electrode 10 described herein can be present as the working electrode, the counter electrode, or both.

Also described herein are electrochemical sensors. According to particular embodiments, the electrochemical sensors may be adapted to detect the presence of, and/or measure the concentration of, an analyte by way of electrochemical oxidation and reduction reactions within the sensor. These reactions can be transduced to an electrical signal that can be correlated to an amount or concentration of the analyte. In certain embodiments, the electrochemical sensor can be a biosensor test strip.

According to still other embodiments, the test strip may include a base substrate, a spacing layer, a covering layer, or any combination thereof. The base substrate can include an electrode system and the electrode system can include a set of measuring electrodes, e.g., at least a working electrode and a counter electrode, within a sample-receiving chamber. According to particular embodiments, one or more of the electrodes in the electrode system can include an electrode as described herein.

Further, in a particular embodiment, the spacing layer of the test strip can define a sample-receiving chamber extending between the base substrate and the covering layer. The sample-receiving chamber can be adapted such that a sample fluid can enter a chamber and be placed in electrolytic contact with both the working electrode and the counter electrode. Such contact can allow electrical current to flow between the measuring electrodes to affect the electrooxidation or electroreduction of the analyte. In very particular embodiments, the sample fluid can be a blood sample, such as a human blood sample, and the sensor can be adapted to measure the glucose level in such a sample.

Moreover, a suitable reagent system can overlie at least a portion of the electrodes or electrode pairs within the sample-receiving chamber. The reagent system can include additives to enhance the reagent properties or characteristics. For example, additives can include materials to facilitate the placement of the reagent composition onto the test strip and to improve its adherence to the strip, or for increasing the rate of hydration of the reagent composition by the sample fluid. Additionally, the additives can include components selected to enhance the physical properties of the resulting dried reagent layer, and the uptake of a liquid test sample for analysis. In certain embodiments, the additives can include thickeners, viscosity modulators, film formers, stabilizers, buffers, detergents, gelling agents, fillers, film openers, coloring agents, agents endowing thixotropy, or any combination thereof.

In further embodiments, the covering layer can be adapted to form a top surface of the sample-receiving chamber. Moreover, the covering layer can be adapted to provide a hydrophilic surface to aid in acquisition of the test sample. In a particular embodiment, the covering layer can define a vent opening that allows air to escape from the interior of the chamber as the sample fluid enters and moves into the sample-receiving chamber.

According to certain embodiments, an electrode as described herein can be formed according to any appropriate method. In general, forming the electrode includes providing a substrate as described herein, depositing the base layer as described herein, and depositing the top layer as described herein. For example, the base layer can be deposited over the substrate and the top layer can be deposited over the base layer. In certain embodiments, the base layer can be deposited directly onto the substrate. In still other embodiments, the top layer can be deposited directly onto the base layer. In another embodiment, the intermediate layer is formed on the base layer and the top layer is deposited directly on the intermediate layer.

According to still other embodiments, the method may include depositing one or more of the layers of the inert, non-noble metal of the base layer, the noble metal of the top layer, or combination thereof by physical vapor deposition, includes sputtering, thermal evaporation, electron beam evaporation, or combination thereof. In an embodiment, the sputtering includes magnetron sputtering in roll-to-roll multiple vacuum chambers, inline multiple vacuum chambers, a stationary co-sputtering vacuum chamber, or combination thereof. In an embodiment, the sputtering is in the presence of an inert gas comprising argon, krypton, xenon, neon, nitrogen, or combination thereof. In a particular embodiment, the inert gas is krypton. According to certain embodiments, the base layer 30 may be deposited with or without annealing. In certain embodiments, the base layer 30 may be deposited without annealing. The materials used for forming the base layer 30 may have any appropriate deposition rate, such as a standard deposition rate.

According to an embodiment, the base layer 30 and the top layer 40 may be deposited by roll-to-roll processing. Roll-to-roll processing refers to a process of applying coatings starting with a roll of a flexible material and re-reeling after the process to create an output roll. In certain embodiments, the roll-to-roll process can include depositing the base and top layers using two sets of cathodes, such as simultaneously using two sets of cathodes.

According to still other embodiments, the base layer 30 may be deposited at a particular pressure of processing gas when using a sputtering process. For example, the base layer 30 may be deposited at a pressure of not greater than about 10 mTorr, such as, not greater than about 9 mTorr, or not greater than about 8 mTorr. According to a particular embodiment, the base layer 30 may be deposited at a pressure of at least about 1 mTorr, at least about 2 mTorr, or at least about 3 mTorr. It will be appreciated that the base layer 30 may be deposited at a pressure of any value within a range between any of the minimum and maximum values noted above. It will be further appreciated that the first layer 30 may be deposited at a pressure of any value between any of the minimum and maximum values noted above.

When present, the intermediate layer 35 may be formed by exposing the base layer to oxygen molecules, oxygen plasma, or combination thereof. Any conditions are envisioned but may include, for example, exposing the base layer to oxygen molecules, which may include oxygen gas under pressure of at least 0.5 mTorr for at least 1 second, or at least 1 mTorr for at least 10 seconds. In an embodiment, exposing the base layer to oxygen plasma may include poisoning a metal target in a chamber under a mixture of oxygen and an inert gas with the ratio of oxygen partial pressure to inert gas partial pressure of at least 0.5% during sputtering.

In yet another embodiment, forming the intermediate layer includes exposing the base layer to oxygen plasma, which includes adding oxygen gas during sputtering of the top layer, i.e. the noble metal layer. In a particular embodiment, the intermediate layer is formed during the sputtering of the top layer under a mixture of inert gas and oxygen. For instance, oxygen gas may be added to krypton gas as a mixture gas during the sputtering of a gold layer. In a more particular embodiment, the standard volume percentage of oxygen gas may be no more than 1% in the mixture.

As described, the top layer 40 may be applied via sputtering. In a particular embodiment, the sputtering of the noble metal layer is in the presence of an inert gas comprising argon, krypton, xenon, neon, nitrogen, or combination thereof. In a particular embodiment, the inert gas is krypton. According to still other embodiments, the top layer 40 may be deposited at a particular pressure. For example, the top layer 40 may be deposited at a pressure of not greater than about 10 mTorr, such as, not greater than about 9 mTorr or not greater than about 8 mTorr. According to particular embodiments, the top layer 40 may be deposited at a pressure of at least about 1 mTorr, at least about 2 mTorr, or at least about 3 mTorr. It will be appreciated that the top layer 40 may be deposited at a pressure of any value within a range between any of the minimum and maximum values noted above. It will be further appreciated that the second layer 40 may be deposited at a pressure of any value between any of the minimum and maximum values noted above.

Many different aspects and embodiments are possible. Some of those aspects and embodiments are described below. After reading this specification, skilled artisans will appreciate that those aspects and embodiments are only illustrative and do not limit the scope of the present invention. Embodiments can be in accordance with any one or more of the items as listed below.

### Embodiment 1

An electrochemical biosensor electrode including: a substrate; a base layer adjacent to the substrate, the base layer including an inert, non-noble metal, the inert, non-noble metal comprising nickel (Ni), chromium (Cr), iron (Fe), manganese (Mn), molybdenum (Mo), an alloy thereof, or combination thereof; and a top layer adjacent to the base layer, the top layer including a noble metal, wherein the top layer has a thickness of not greater than 15 nanometers (nm).

### Embodiment 2

A electrochemical biosensor electrode including: a substrate; a base layer adjacent to the substrate, the base layer including an inert, non-noble metal, the inert, non-noble metal comprising nickel (Ni), chromium (Cr), iron (Fe), manganese (Mn), molybdenum (Mo), an alloy thereof, or combination thereof; an intermediate layer adjacent to the base layer, the intermediate layer comprising an inert, non-noble metal oxide; and a top layer adjacent to the intermediate layer, wherein the top layer comprises a noble metal having a thickness of not greater than 15 nanometers (nm).

### Embodiment 3

A method of forming an electrochemical biosensor electrode including: providing a substrate; depositing a base layer on the substrate, wherein the base layer includes an inert, non-noble metal, the inert, non-noble metal including nickel (Ni), chromium (Cr), iron (Fe), manganese (Mn), molybdenum (Mo), an alloy thereof, or combination thereof; optionally forming an intermediate layer on the base layer, wherein the intermediate layer comprises an inert non-noble metal oxide; and depositing a top layer adjacent to the base layer, the top layer including a noble metal, the top layer having a thickness of not greater than 15 nanometers (nm).

### Embodiment 4

The electrochemical biosensor electrode or method of forming the electrochemical biosensor electrode in accordance with any of the preceding embodiments, wherein the inert, non-noble metal comprises an alloy of nickel (Ni) and chromium (Cr), an alloy of nickel, chromium, and iron (Fe), or combination thereof.

### Embodiment 5

The electrochemical biosensor electrode or method of forming the electrochemical biosensor electrode in accordance with embodiment 4, wherein the inert, non-noble metal includes an alloy of nickel, chromium, and iron.

### Embodiment 6

The electrochemical biosensor electrode or method of forming the electrochemical biosensor electrode in accordance with any of the preceding embodiments, wherein the base layer has a thickness of at least 5 nm, such as at least 10 nm, at least 15 nm, at least 20 nm, at least 25 nm, at least 30 nm, at least 35 nm, or at least 40 nm.

### Embodiment 7

The electrochemical biosensor electrode or method of forming the electrochemical biosensor electrode in accordance with embodiment 6, wherein the base layer has a thickness of not greater than 200 nm, such as not greater than 190 nm, not greater than 180 nm, not greater than 170 nm, not greater than 160 nm, or not greater than 150 nm.

### Embodiment 8

The electrochemical biosensor electrode or method of forming the electrochemical biosensor electrode in accordance with any of the preceding embodiments, wherein the noble metal includes gold, palladium, platinum, iridium, ruthenium, an alloy, or combination thereof.

### Embodiment 9

The electrochemical biosensor electrode or method of forming the electrochemical biosensor electrode in accordance with any of the preceding embodiments, wherein the top layer further includes a trace element of sputtering gas including argon, krypton, xenon, neon, nitrogen, or combination thereof.

### Embodiment 10

The electrochemical biosensor electrode or method of forming the electrochemical biosensor electrode in accordance with embodiment 9, wherein the trace element of sputter gas includes krypton.

### Embodiment 11

The electrochemical biosensor electrode or method of forming the electrochemical biosensor electrode in accordance with embodiment 9, wherein the trace element is present at an amount of at least 1×10⁵ atoms/cm³, such as at least 1×10¹⁰ atoms/cm³.

### Embodiment 12

The electrochemical biosensor electrode or method of forming the electrochemical biosensor electrode in accordance with any of the preceding embodiments, wherein the top layer consists essentially of the noble metal and krypton.

### Embodiment 13

The electrochemical biosensor electrode or method of forming the electrochemical biosensor electrode in accordance with embodiment 12, wherein the noble metal consists essentially of gold, palladium, or combination thereof.

### Embodiment 14

The electrochemical biosensor electrode or method of forming the electrochemical biosensor electrode in accordance with any of the preceding embodiments, wherein top layer has a thickness of at least 2 nm, such as at least 3 nm, at least 4 nm, or at least 5 nm.

### Embodiment 15

The electrochemical biosensor electrode or method of forming the electrochemical biosensor electrode in accordance with any of the preceding embodiments, wherein the top layer has a thickness of not greater than 14 nm, such as not greater than 13 nm, not greater than 12 nm, or not greater than 11 nm.

### Embodiment 16

The electrochemical biosensor electrode or method of forming the electrochemical biosensor electrode in accordance with embodiment 1, further including an intermediate layer directly in contact with the base layer and the top layer, wherein the intermediate layer includes an inert, non-noble metal oxide.

### Embodiment 17

The biosensor electrode or method of forming the biosensor electrode in accordance with any of the preceding embodiments, wherein the inert, non-noble metal oxide includes an oxide of: nickel (Ni), nickel alloy, chromium (Cr), chromium alloy, iron (Fe), iron alloy, manganese (Mn), manganese alloy, molybdenum (Mo), molybdenum alloy, or combination thereof.

### Embodiment 18

The electrochemical biosensor electrode or method of forming the electrochemical biosensor electrode in accordance with embodiment 17, wherein the inert, non-noble metal oxide includes a nickel oxide, a chromium oxide, an iron oxide, or combination thereof.

### Embodiment 19

The electrochemical biosensor electrode or method of forming the electrochemical biosensor electrode in accordance with any of the preceding embodiments, wherein the intermediate layer has a thickness of at least 0.5 nm, such as at least 0.75 nm, at least 1.0 nm, at least 1.25 nm, at least 1.5 nm, at least 1.75 nm, or at least 2.0 nm.

### Embodiment 20

The electrochemical biosensor electrode or method of forming the electrochemical biosensor electrode in accordance with any of the preceding embodiments, wherein the intermediate layer has a thickness of not greater than 5.0 nm, such as not greater than 4.75 nm, not greater than 4.5 nm, not greater than 4.25 nm, or not greater than 4.0 nm.

### Embodiment 21

The electrochemical biosensor electrode or method of forming the electrochemical biosensor electrode in accordance with any of the preceding embodiments, wherein the substrate includes a polymer, a flexible polymer, or a transparent polymer.

### Embodiment 22

The electrochemical biosensor electrode or method of forming the electrochemical biosensor electrode in accordance with embodiment 21, wherein the substrate includes polycarbonate, polyacrylate, polyester, polyethylene terephthalate (PET), polyethylene naphthalate (PEN), cellulose triacetated (TCA or TAC), polyurethane (PU), or any combination thereof.

### Embodiment 23

The electrochemical biosensor electrode or method of forming the electrochemical biosensor electrode in accordance with embodiment 22, wherein the substrate includes polyethylene terephthalate (PET).

### Embodiment 24

The electrochemical biosensor electrode or method of forming the electrochemical biosensor electrode in accordance with any one of the preceding embodiments, wherein the substrate has a thickness of at least 25 microns (μm), such as at least 30 microns, at least 35 microns, at least 40 microns, at least 45 microns, or at least 50 microns.

### Embodiment 25

The electrochemical biosensor electrode or method of forming the electrochemical biosensor electrode in accordance with any one of the preceding embodiments, wherein the substrate has a thickness of not greater than 350 microns, such as not greater than 325 microns, not greater than 300 microns, or not greater than 275 microns.

### Embodiment 26

The electrochemical biosensor electrode or method of forming the electrochemical biosensor electrode in accordance with embodiment 1, wherein the base layer directly contacts the substrate.

### Embodiment 27

The electrochemical biosensor electrode or method of forming the electrochemical biosensor electrode in accordance with embodiment 26, wherein the top layer directly contacts the base layer.

### Embodiment 28

The electrochemical biosensor electrode or method of forming the electrochemical biosensor electrode in accordance with any of the preceding embodiments, wherein the electrode measures glucose in blood.

### Embodiment 29

The method of forming the electrochemical biosensor electrode in accordance with any of the preceding embodiments, wherein depositing the base layer, the top layer, or combination thereof is via a physical vapor deposition.

### Embodiment 30

The method of forming the electrochemical biosensor electrode in accordance with embodiment 29, wherein the physical vapor deposition includes sputtering, thermal evaporation, electron beam evaporation, or combination thereof.

### Embodiment 31

The method of forming the electrochemical biosensor electrode in accordance with embodiment 30, wherein sputtering includes magnetron sputtering in roll-to-roll multiple vacuum chambers, inline multiple vacuum chambers, a stationary co-sputtering vacuum chamber, or combination thereof.

### Embodiment 32

The method of forming the electrochemical biosensor electrode in accordance with embodiment 31, wherein the sputtering is in the presence of an inert gas including argon, krypton, xenon, neon, nitrogen, or combination thereof.

### Embodiment 33

The method of forming the electrochemical biosensor electrode in accordance with embodiment 32, wherein the inert gas includes krypton.

### Embodiment 34

The method of forming the electrochemical biosensor electrode in accordance with any of the preceding embodiments, wherein forming the intermediate layer includes exposing the base layer to oxygen molecules, oxygen plasma, or combination thereof.

### Embodiment 35

The method of forming the electrochemical biosensor electrode in accordance with embodiment 34, wherein exposing the base layer to oxygen molecules includes oxygen gas under pressure of at least 0.5 mTorr for at least 1 second, or at least 1 mTorr for at least 10 seconds.

### Embodiment 36

The method of forming the electrochemical biosensor electrode in accordance with embodiment 34, wherein exposing the base layer to oxygen plasma includes poisoning a metal target in a chamber under a mixture of oxygen and an inert gas with the ratio of oxygen partial pressure to inert gas partial pressure of at least 0.5% during sputtering.

### Embodiment 37

The method of forming the electrochemical biosensor electrode in accordance with embodiment 34, wherein exposing the base layer to oxygen plasma includes adding oxygen gas during sputtering of the top layer.

The following examples are provided to better disclose and teach processes and compositions of the present invention. They are for illustrative purposes only, and it must be acknowledged that minor variations and changes can be made without materially affecting the spirit and scope of the invention as recited in the claims that follow.

### EXAMPLES

### Construction of Samples:

A series of samples were made under different conditions in roll-to-roll magnetron sputtering coater machines. The inert non-noble metal base layer was nickel chrome alloy with nickel and chrome as two major elements or stainless-steel alloy with iron as major element. The alloys used were stainless steel and nickel chromium with the primary elements in these alloys listed in Table 1.

TABLE 1

| | Primary Element (% by mass) | | | | | |
|---|---|---|---|---|---|---|
| Alloy | Fe | Ni | Cr | Mo | Mn | C |
| Stainless | 61.8 to 72 | 10 to 14 | 16 to 18 | 2 to 3 | ≤2 | ≤0.08 |
| Steel-1 | | | | | | |
| Stainless | 61.8 to 72 | 10 to 14 | 16 to 18 | 2 to 3 | ≤2 | ≤0.03 |
| Steel-2 | | | | | | |
| Stainless | 66.3 to 74 | 8 to 10.5 | 17 to 20 | — | ≤2 | ≤0.08 |
| Steel-3 | | | | | | |
| NiCr-1 | — | 80 | 20 | — | — | — |
| NiCr-2 | 6 to 10 | ≥72 | 14 to 17 | — | ≤0.5 | ≤0.15 |

As embodiments, Stainless Steel-1 were deposited on a flexible PET film substrate from the DuPont Teijin Films by using rotary magnetron cathodes and a noble metal layer such as gold and palladium was deposited on top of the stainless-steel base layer by using planar magnetron cathodes. The flexible PET film substrate had a thickness of 10 mil and surface roughness Ra (arithmetic average) of about 130 nm. The substrate was introduced to the coater in a roll format and the coating layers were deposited on it or processed continuously under different process parameters for different samples. The thickness of noble metal and inert non-noble metal were controlled by sputtering power and the speed at which PET film was run through the coaters or cathodes.

The noble metal layer was deposited in either Argon or Krypton sputtering gas and the base layer was deposited in Argon sputtering gas. The intermediate layer was formed by directly oxidizing the inert non-noble metal base layer in a glow discharging (G.D.) chamber source in either DC (direct current) or AC mode (alternating current) where oxygen plasma was generated in a mixture gas of oxygen and argon, or, optionally, it was followed by further oxidization in an oxidization chamber with oxygen gas. The thickness of the intermediate layer was controlled by the power (KW) of glow discharge, oxygen gas flow (sccm) and speed (foot per minute or fpm) for these samples.

The conductive performance of the composite film was characterized by measuring sheet resistance (Ohm/sq) using a four-terminal sensing measurement (also known as a four-point probe measurement). The coating thickness (nanometer or nm) of noble or inert non-noble metal layer was measured using XRF (X-ray Fluorescence) method.

Scratch resistance is a measure of the composite film ability to resist scratching. Although not being bound by theory, the scratch resistance of an electrode may be relevant to the performance of a biosensor device because, when used, a biosensor electrode may be inserted into a digital reader system and the electrode may perform an electrical contact with metallic pins in the digital reader system. If the metallic pins create a scratch on the biosensor electrode when the biosensor is inserted into the digital reader, false readings or a non-measurement may occur. The scratch resistance is also relevant to manufacture process of biosensor electrode because the composite film must go through many process steps with conductive surface contacting on various processing surfaces, which can potentially generate scratches. These scratches could affect the production yield if they are detected during manufacture step, or false readings or a non-measurement could occur if they are not detected and used in a biosensor device. Scratch resistance is measured using an Erichsen Hardness Test Pencil, for example model 308S. The conductive layer of the composite film was scratched using the pencil under a 0.6 N load. The scratch resistance for the conductive composite was reported as the width of the scratch.

To quantify the electrochemical performance of a conductive composite films, widely used Cyclic Voltammetry (CV) scans were performed, and the results were analyzed to determine shifts in the current density and potential of oxidation peaks. CV experiments were conducted in the Potassium Phosphate buffer solution (pH=7.4) with 5 mM potassium ferricyanide and 145 mM sodium chloride. In some cases, both 5 mM potassium ferricyanide and 5 mM potassium ferrocyanide were used. Electrode samples were masked by an electroplating tape with 5 mm diameter opening as an active area. Three electrode system was used with the masked samples as working electrode, Pt wire as the counter electrode, and Ag/AgCl as a reference electrode. CV was performed by scanning the potential of the working electrode (biosensor composite film) between-0.15 V and 0.6 V with reference to an Ag/AgCl reference electrode at different scan rates from 20 mV/s to 1 V/s. During the scanning, responding currents were measured and the CV results are shown in figures. CV oxidation peak position and/or height (current) was determined and compared to evaluate the biosensor sensitivity. In some cases, Differential Pulse Voltammetry (DPV) on some of the Au samples under the same conditions were performed. DPV was more sensitive and accurate than CV because it effectively eliminated the contributions from the background current and capacitor charging current during the CV scans.

Secondary ion mass spectrometry (SIMS) was employed to detect and quantify the formation of an oxide passivation layer at the interface between noble metal and inert non-noble metal. SIMS depth profiling was performed using a TOF.SIMS 5 instrument (IONTOF GmbH, Münster, Germany). Cesium ions (Cs⁺) were selected as the sputtering species. The sputter beam operated at an energy of 2 keV with a current of 170 nA. The analysis beam, used in pulsed mode for time-of-flight mass spectrometry, consisted of Bi₃⁺ ions at 30 keV with a current of 1.2 pA. The scanned area measured 100×100 μm², centered within a sputtered zone of 300×300 μm². To minimize charging effects, a low-energy electron flood gun was employed.

Evidence of the oxide passivation layer was obtained by comparing SIMS profiles across different samples. It is noteworthy that monoatomic ions (M⁺) are more sensitive in oxidized matrices, whereas cluster ions (CsM⁺) are more sensitive in reduced matrices. Therefore, the presence of the oxide passivation layer was identified by monitoring the signals of Fc⁺, Cr⁺, Ni⁺, and Cs₂O⁺ across the samples. Although SIMS profiles are commonly plotted on a logarithmic scale, the oxide passivation layer was even more apparent when visualized on a linear scale.

Secondary ion mass spectrometry (SIMS) is employed to measure the concentration of sputtering inert gas (Argon or Krypton) included inside sputtered noble metal.

### Example 1

A series of conductive composite films with gold as the noble metal were created. The samples were then tested for various properties including sheet resistance, layer thickness and electrochemical performance.

The deposition parameters, sheet resistance and layer thickness are listed in Table 2. The coater used for sputtering gold and G.D. in AC mode had an effective width about 1.5 meters.

TABLE 2

| | | | | | Passivation Process | | | |
|---|---|---|---|---|---|---|---|---|
| | | | | Stainless | G.D. | G.D. | G.D. | Oxidation |
| | | Au | Au | steel -1 | AC | Ar | O2 | Chamber |
| Sample | Rs | Sputtering | Thickness | Thickness | Power | Flow | Flow | O2 Flow |
| ID | (Ohm/sq) | Gas | (nm) | (nm) | (kW) | (sccm) | (sccm) | (sccm) |
| A1 | 4 | Argon | 20 | N.A. | N.A. | N.A. | N.A. | N.A. |
| A2 | 10 | Argon | 10 | N.A. | N.A. | N.A. | N.A. | N.A. |
| A3 | 7 | Argon | 10 | 50 | 0.0 | 1600 | 0 | 0 |
| A4 | 7 | Argon | 10 | 50 | 3.0 | 1600 | 200 | 4000 |
| A5 | 5 | Krypton | 10 | 50 | 3.0 | 1600 | 200 | 4000 |

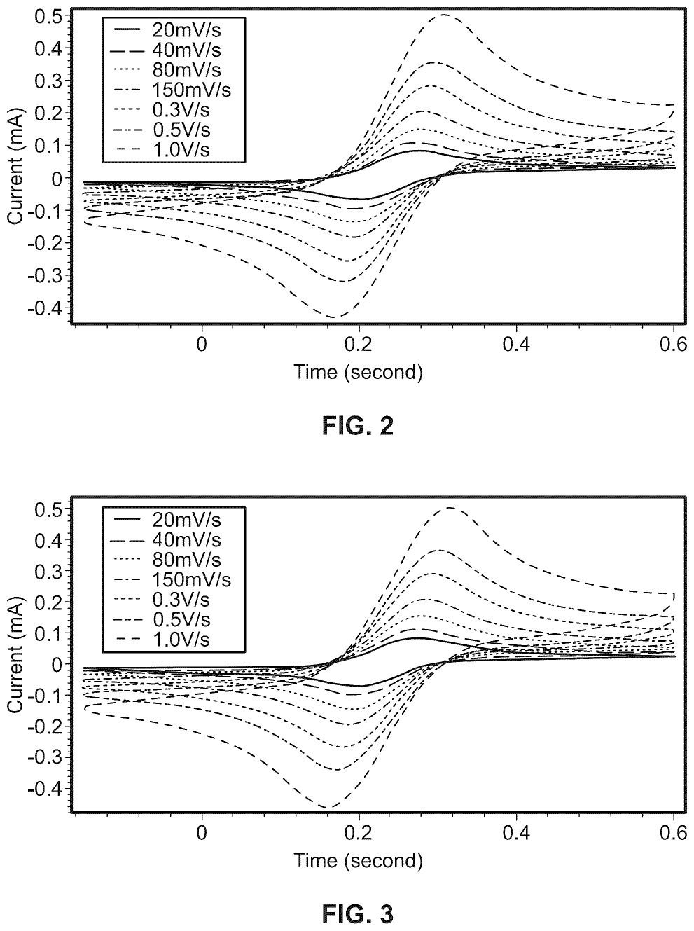

The samples were stored in vacuum-sealed aluminum bag and then exposed to ambient environment for less than one month before completing electrochemical measurements. The electrochemical performance was measured by CV scans in 5 mM ferrocyanide solution at different scan rates from 20 mV/sec to 1V/sec. CV scans were plotted and can be seen in FIG. 2 to FIG. 5.

FIG. 2 illustrates CV scans of Sample A1 that was 20 nm pure gold reference sample in potassium phosphate buffer solution (pH=7.4) with 5 mM potassium ferricyanide and 145 mM sodium chloride at scan rates from 20 mV/sec to 1V/sec.

FIG. 3 illustrates CV scans of Sample A2 that was 10 nm pure gold reference sample in potassium phosphate buffer solution (pH=7.4) with 5 mM potassium ferricyanide and 145 mM sodium chloride at scan rates from 20 mV/sec to 1V/sec.

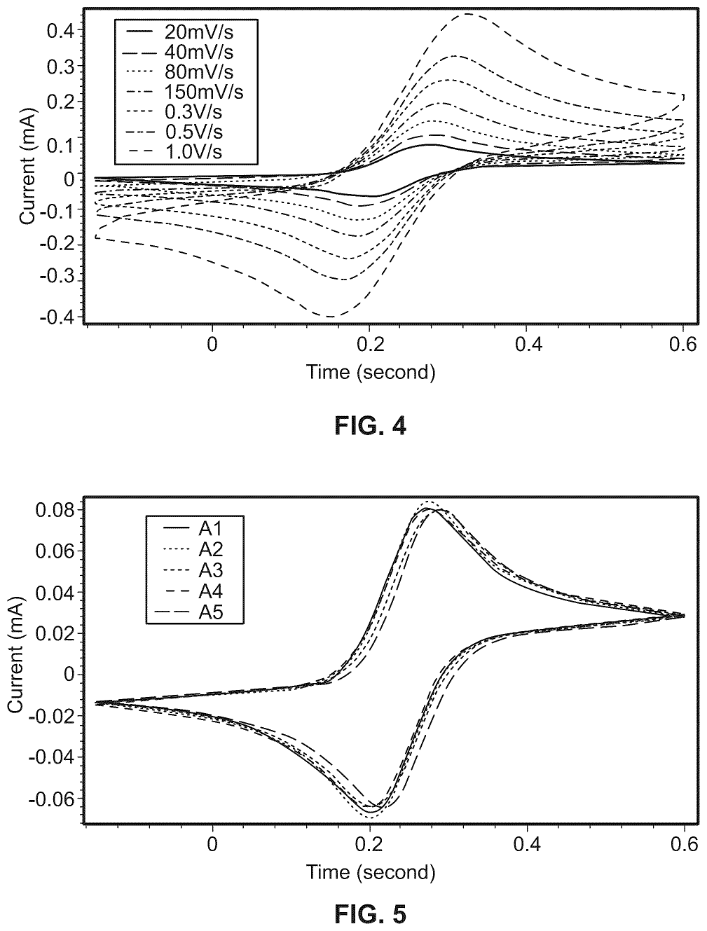

FIG. 4 illustrates CV scans of Sample A4 that was 10 nm gold on passivated Stainless Steel-1 by oxygen in potassium phosphate buffer solution (pH=7.4) with 5 mM potassium ferricyanide and 145 mM sodium chloride at scan rates from 20 mV/sec to 1V/sec. Sample A3 had comparable CV scans with pure gold reference samples.

FIG. 5 illustrates CV scans of Samples A1 to A5 in potassium phosphate buffer solution (pH=7.4) with 5 mM potassium ferricyanide and 145 mM sodium chloride at scan rates of 20 m V/sec.

### Example 2

A series of conductive composite films with gold as the noble metal were created to investigate the effects of passivation layer and type of sputtering gas. The samples were then tested for various properties including sheet resistance, layer thickness and electrochemical performance.

The deposition parameters, sheet resistance and layer thickness are listed in Table 3. The coater used for sputtering gold and G.D. in AC mode has an effective width about 1.5 meters.

TABLE 3

| | | | | | Passivation Process | | | |
|---|---|---|---|---|---|---|---|---|
| | | | | Stainless | G.D. | G.D. | G.D. | Oxidation |
| | | Au | Au | steel -1 | AC | Ar | O2 | Chamber |
| Sample | Rs | Sputtering | Thickness | Thickness | Power | Flow | Flow | O2 Flow |
| ID | (Ohm/sq) | Gas | (nm) | (nm) | (kW) | (sccm) | (sccm) | (sccm) |
| B1 | 15 | Ar | 7 | N.A. | N.A. | N.A. | N.A. | N.A. |
| B2 | 13 | Ar | 7 | 20 | 3 | 1600 | 200 | 4000 |
| B3 | 19 | Ar | 5 | 20 | 3 | 1600 | 200 | 4000 |
| B4 | 14 | Ar | 7 | 20 | 0 | 1600 | 0 | 0 |
| B5 | 20 | Ar | 5 | 20 | 0 | 1600 | 0 | 0 |
| B6 | 11 | Kr | 7 | 20 | 3 | 1600 | 200 | 4000 |
| B7 | 19 | Kr | 5 | 20 | 3 | 1600 | 200 | 4000 |
| B8 | 55 | Kr | 3 | 20 | 3 | 1600 | 200 | 4000 |
| B9 | 12 | Kr | 7 | 20 | 0 | 1600 | 0 | 0 |
| B10 | 18 | Kr | 5 | 20 | 0 | 1600 | 0 | 0 |
| B11 | 50 | Kr | 3 | 20 | 0 | 1600 | 0 | 0 |

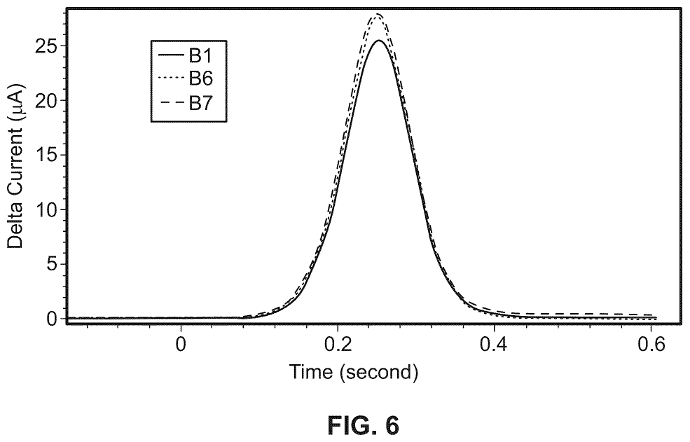

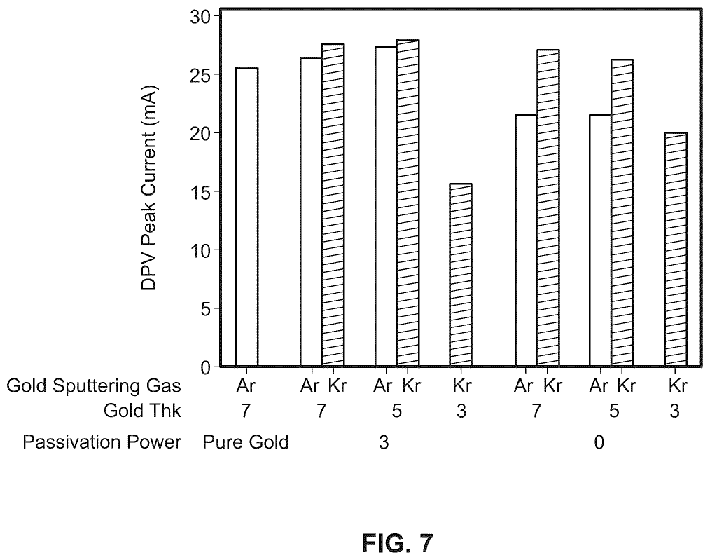

The samples were exposed to ambient environment for about 6 months before the electrochemical measurements. The electrochemical performance was measured by DPV scans in potassium phosphate buffer solution (pH=7.4) with 5 mM potassium ferricyanide and 145 mM sodium chloride. The results were plotted in FIG. 6 and FIG. 7.

FIG. 6 illustrates typical DPV scans of Sample B1, Sample B6 and Sample B7 in potassium phosphate buffer solution (pH=7.4) with 5 mM potassium ferricyanide and 145 mM sodium chloride.

FIG. 7 illustrates DPV peak currents of all samples in Table 3. As illustrated, the conductive composite films with gold sputtered in krypton gas and gold thickness of 5 nm or larger had the highest peak currents. The peak currents degraded at 3 nm gold thickness. The composite films with gold sputtered in krypton had higher peak currents than those sputtered in argon.

### Example 3

A series of conductive composite films with gold as the noble metal were created to investigate the effect of base Stainless Steel-1 layer on scratching resistance. The deposition parameters and layer thickness are listed in Table 4. The coater used for sputtering gold and G.D. in AC mode had an effective width about 1.5 meters.

TABLE 4

| | | | | Passivation Process | | | |
|---|---|---|---|---|---|---|---|
| | | | Stainless | G.D. | G.D. | G.D. | Oxidation |
| | Au | Au | steel -1 | AC | Ar | O2 | Chamber |
| Sample | Sputtering | Thickness | Thickness | Power | Flow | Flow | O2 Flow |
| ID | Gas | (nm) | (nm) | (kW) | (sccm) | (sccm) | (sccm) |
| C1 | Krypton | 23 | N.A. | N.A. | N.A. | N.A. | N.A. |
| C2 | Argon | 7 | N.A. | N.A. | N.A. | N.A. | N.A. |
| C3 | Argon | 4 | 50 | 0.0 | 0 | 0 | 0 |
| C4 | Argon | 5 | 20 | 3.0 | 1600 | 200 | 4000 |
| C5 | Argon | 5 | 20 | 0.0 | 1600 | 0 | 0 |
| C6 | Krypton | 5 | 20 | 3.0 | 1600 | 200 | 4000 |
| C7 | Krypton | 5 | 20 | 0.0 | 1600 | 0 | 0 |

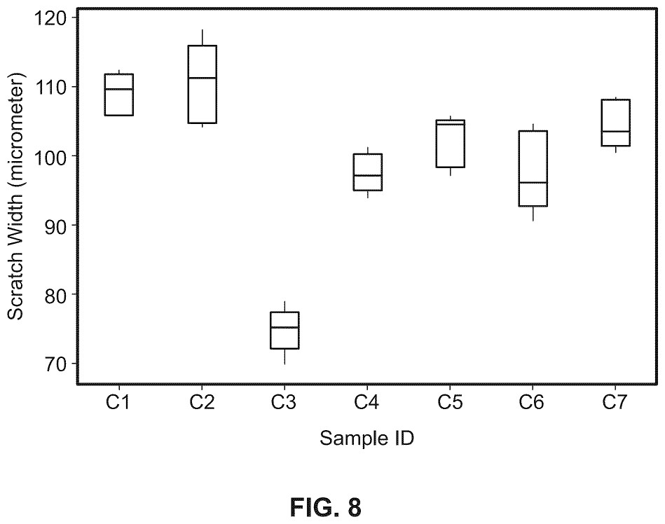

FIG. 8 illustrates the scratching width of all samples in Table 4. As illustrated, 50 nm Stainless Steel-1 significantly reduced the scratching width and 20 nm Stainless Steel-1 has the similar effect but in much less magnitude.

### Example 4

SIMS analysis was performed to investigate the formation of oxide at the interface between noble metal and inert non-noble metal of A3 and A4 samples in Example 1. Because of oxidation of samples in air, oxygen signal was observed at the interface between gold and stainless steel 316 in A3 and A4, but higher oxygen signal was observed for A4 where the stainless steel 316 was passivated with G.D. during sputtering. The oxidized portion was estimated to be approximately 4-5 nm.

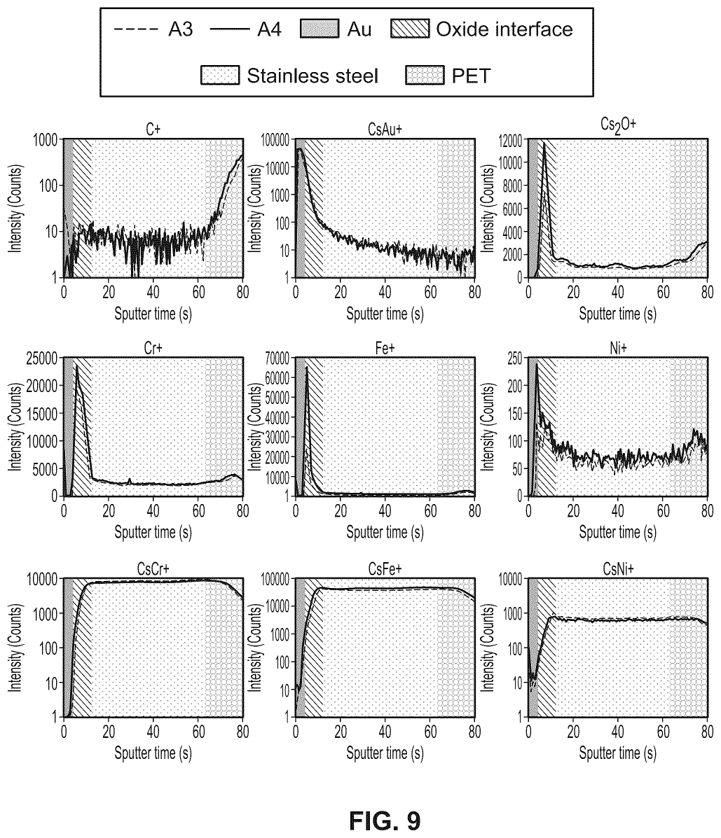

FIG. 9 illustrates SIMS analysis results.

### Example 5

SIMS analysis is performed to investigate the concentration of Krypton included in the noble metal. The concentration of Krypton present in the gold layer is at least about 1×10¹⁰ atoms/cm³.

### Example 6

Two conductive composite film samples with Palladium (Pd) as the noble metal were created. The samples were then tested for various properties including sheet resistance, layer thickness and electrochemical performance.

The deposition parameters, sheet resistance and layer thickness are listed in Table 5. The coater used for sputtering Palladium and G.D. in DC mode has an effective width about 1.2 meters. No oxidation chamber was available in this coater.

TABLE 5

| | | | | | Passivation Process | | | |
|---|---|---|---|---|---|---|---|---|
| | | | | Stainless | G.D. | G.D. | G.D. | Oxidation |
| | | Pd | Pd | steel -1 | DC | Ar | O2 | Chamber |
| Sample | Rs | Sputtering | Thickness | Thickness | Power | Flow | Flow | O2 Flow |
| ID | (Ohm/sq) | Gas | (nm) | (nm) | (kW) | (sccm) | (sccm) | (sccm) |
| D1 | 13 | Argon | 22 | N.A. | N.A. | N.A. | N.A. | N.A. |
| D2 | 12 | Argon | 6 | 70. | 2.0 | 1200 | 100 | N.A. |

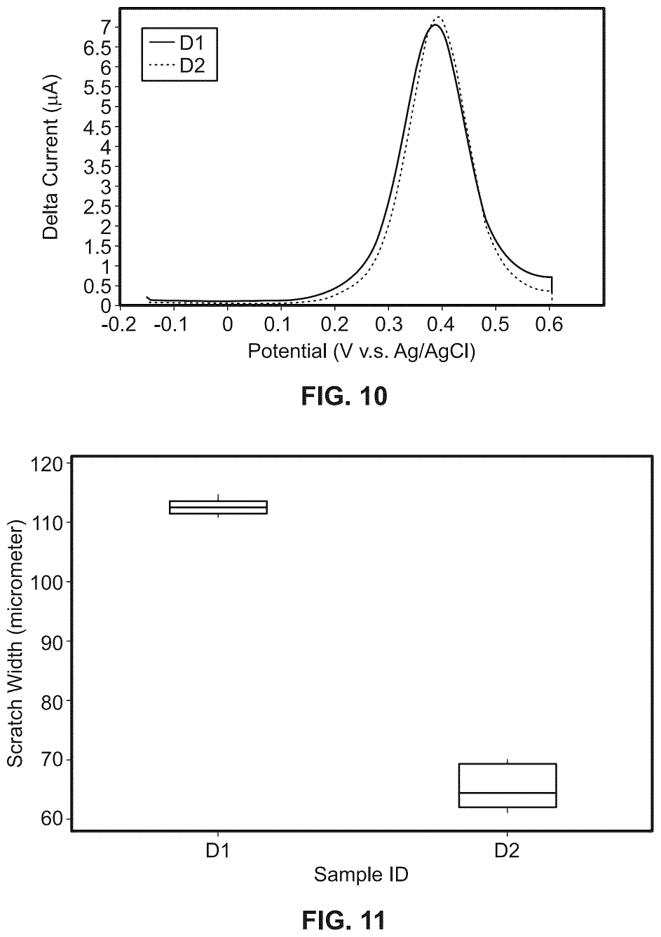

FIG. 10 illustrates DPV scans of Samples D1 and D2 in potassium phosphate buffer solution (pH=7.4) with 5 mM potassium ferricyanide and 145 mM sodium chloride. Sample D2 had comparable DPV scans with pure Pd reference sample (D1).

FIG. 11 illustrates the scratching width of all samples in Table 5. As illustrated, 70 nm Stainless Steel-1 significantly reduced the scratching width compared to 22 nm pure Palladium sample.

Note that not all of the activities described above in the general description or the examples are required, that a portion of a specific activity cannot be required, and that one or more further activities can be performed in addition to those described. Still further, the order in which activities are listed is not necessarily the order in which they are performed.

Benefits, other advantages, and solutions to problems have been described above with regard to specific embodiments. However, the benefits, advantages, solutions to problems, and any features that can cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments can also be provided in combination in a single embodiment, and conversely, various features that are, for brevity, described in the context of a single embodiment, can also be provided separately or in any subcombination. Further, reference to values stated in ranges includes each and every value within that range. Many other embodiments can be apparent to skilled artisans only after reading this specification. Other embodiments can be used and derived from the disclosure, such that a structural substitution, logical substitution, or another change can be made without departing from the scope of the disclosure. Accordingly, the disclosure is to be regarded as illustrative rather than restrictive.

What is claimed is:

1. An electrochemical biosensor electrode comprising:
a substrate;
a base layer adjacent to the substrate, the base layer comprising an inert, non-noble metal, the inert, non-noble metal comprising nickel (Ni), chromium (Cr), iron (Fe), manganese (Mn), molybdenum (Mo), an alloy thereof, or combination thereof; and
a top layer adjacent to the base layer, the top layer comprising a noble metal, wherein the top layer has a thickness of not greater than 15 nanometers (nm).

2. The electrochemical biosensor electrode in accordance with claim 1, wherein the inert, non-noble metal comprises an alloy of nickel (Ni) and chromium (Cr), an alloy of nickel, chromium, and iron (Fe), or a combination thereof.

3. The electrochemical biosensor electrode in accordance with claim 1, wherein the base layer has a thickness of at least 5 nm, such as at least 10 nm, at least 15 nm, at least 20 nm, at least 25 nm, at least 30 nm, at least 35 nm, or at least 40 nm and not greater than 200 nm, such as not greater than 190 nm, not greater than 180 nm, not greater than 170 nm, not greater than 160 nm, or not greater than 150 nm.

4. The electrochemical biosensor electrode in accordance with claim 1, wherein the noble metal comprises gold, palladium, platinum, iridium, ruthenium, an alloy, or combination thereof.

5. The electrochemical biosensor electrode in accordance with claim 1, wherein the top layer further comprises a trace element of sputtering gas comprising argon, krypton, xenon, neon, nitrogen, or combination thereof.

6. The electrochemical biosensor electrode in accordance with claim 1, wherein top layer has a thickness of at least 2 nm, such as at least 3 nm, at least 4 nm, or at least 5 nm and not greater than 14 nm, such as not greater than 13 nm, not greater than 12 nm, or not greater than 11 nm.

7. The electrochemical biosensor electrode in accordance with claim 1, further comprising an intermediate layer directly in contact with the base layer and the top layer, wherein the intermediate layer comprises an inert, non-noble metal oxide.

8. The electrochemical biosensor electrode in accordance with claim 1, wherein the inert, non-noble metal oxide comprises an oxide of: nickel (Ni), nickel alloy, chromium (Cr), chromium alloy, iron (Fe), iron alloy, manganese (Mn), manganese alloy, molybdenum (Mo), molybdenum alloy, or combination thereof.

9. The electrochemical biosensor electrode in accordance with claim 8, wherein the inert, non-noble metal oxide comprises a nickel oxide, a chromium oxide, an iron oxide, or combination thereof.

10. The electrochemical biosensor electrode in accordance with claim 1, wherein the intermediate layer has a thickness of at least 0.5 nm, such as at least 0.75 nm, at least 1.0 nm, at least 1.25 nm, at least 1.5 nm, at least 1.75 nm, or at least 2.0 nm and not greater than 5.0 nm, such as not greater than 4.75 nm, not greater than 4.5 nm, not greater than 4.25 nm, or not greater than 4.0 nm.

11. The electrochemical biosensor electrode in accordance with claim 1, wherein the substrate comprises a polymer, a flexible polymer, or a transparent polymer.

12. The electrochemical biosensor electrode in accordance with claim 11, wherein the substrate comprises polycarbonate, polyacrylate, polyester, polyethylene terephthalate (PET), polyethylene naphthalate (PEN), cellulose triacetated (TCA or TAC), polyurethane (PU), or any combination thereof.

13. The electrochemical biosensor electrode in accordance with claim 1, wherein the substrate has a thickness of at least 25 microns (μm), such as at least 30 microns, at least 35 microns, at least 40 microns, at least 45 microns, or at least 50 microns and not s than 350 microns, such as not greater than 325 microns, not greater than 300 microns, or not greater than 275 microns.

14. The electrochemical biosensor electrode in accordance with claim 1, wherein the electrode measures glucose in blood.

15. A electrochemical biosensor electrode comprising:
a substrate;
a base layer adjacent to the substrate, the base layer comprising an inert, non-noble metal, the inert, non-noble metal comprising nickel (Ni), chromium (Cr), iron (Fe), manganese (Mn), molybdenum (Mo), an alloy thereof, or combination thereof;
an intermediate layer adjacent to the base layer, the intermediate layer comprising an inert, non-noble metal oxide; and
a top layer adjacent to the intermediate layer, wherein the top layer comprises a noble metal having a thickness of not greater than 15 nanometers (nm).

16. A method of forming an electrochemical biosensor electrode comprising:
providing a substrate;
depositing a base layer on the substrate, wherein the base layer comprises an inert, non-noble metal, the inert, non-noble metal comprising nickel (Ni), chromium (Cr), iron (Fe), manganese (Mn), molybdenum (Mo), an alloy thereof, or combination thereof;
optionally forming an intermediate layer on the base layer, wherein the intermediate layer comprises an inert non-noble metal oxide; and
depositing a top layer adjacent to the base layer, the top layer comprising a noble metal, the top layer having a thickness of not greater than 15 nanometers (nm).

17. The method of forming the electrochemical biosensor electrode in accordance with claim 16, wherein depositing the base layer, the top layer, or combination thereof is via a physical vapor deposition.

18. The method of forming the electrochemical biosensor electrode in accordance with claim 17, wherein the physical vapor deposition comprises sputtering, thermal evaporation, electron beam evaporation, or combination thereof.

19. The method of forming the electrochemical biosensor electrode in accordance with claim 18, wherein the sputtering is in the presence of an inert gas comprising argon, krypton, xenon, neon, nitrogen, or combination thereof.

20. The method of forming the electrochemical biosensor electrode in accordance with claim 16, wherein forming the intermediate layer comprises exposing the base layer to oxygen molecules, oxygen plasma, or combination thereof.
